(19) United States
(12) Patent Application Publication
(10) Pub. No.: US 2025/0333776 A1
(43) Pub. Date: Oct. 30, 2025

(54) DETERMINATION OF ANTIMICROBIAL SUSCEPTIBILITY BY POPULATION PROFILING OF MICROBIAL CULTURES

(71) Applicant: Affinity Biosensors, LLC, Santa Barbara, CA (US)

(72) Inventors: Kenneth Babcock, Santa Barbara, CA (US); Cynthia Schneider, Santa Barbara, CA (US); John Tedesco, Santa Barbara, CA (US); Stephen Strenn, Santa Barbara, CA (US); Zachary Ruhe, Goleta, CA (US)

(21) Appl. No.: 19/187,756

(22) Filed: Apr. 23, 2025

Related U.S. Application Data
(60) Provisional application No. 63/638,274, filed on Apr. 24, 2024.

(51) Int. Cl. C12Q 1/20; C12M 1/32; C12M 1/34; C12M 3/06

(52) U.S. Cl. CPC C12Q 1/20; C12M 23/12; C12M 23/16; C12M 41/46

(57) ABSTRACT

Systems and Methods for determining response of microbe cultures to antimicrobials by measuring properties of individual microbes and/or other culture particles. Methods include incubating microbes in liquid cultures which may contain antimicrobials and growth media, detecting individual microbes and particles, and measuring their properties, which may include the mass of individual microbes and particles. Measurement data, such as masses of a plurality of individual microbes or particles, may be collected after an incubation duration. These data may be analyzed to determine susceptibility of microbes to one or more antibiotics. Measurement may be performed after incubation durations shorter than required to produce growth by microbe replication, and analysis may include assessment of metrics other than the growth of the culture. The method enables measurement of susceptibility at earlier times than needed for sufficient microbe replication required by many existing techniques enabling accurate results with relatively minimal sample volumes of a culture.

### RELATED APPLICATIONS

This application claims priority to U.S. Provisional Application Ser. No. 63/638,274 filed Apr. 24, 2024 whose contents are incorporated by reference in their entirety

### BACKGROUND

Antimicrobial susceptibility testing is widely used in-vitro diagnostic that determines which antimicrobials will be effective when treating patients with bacterial and fungal infections. This disclosure allows susceptibility results to be produced faster than with currently employed methods, reducing the time to effective treatment. The specification relates to methods and systems for determining antimicrobial susceptibility from properties other than, or in addition to, aggregate growth of microbial cultures.

### SUMMARY

Example embodiments described herein have innovative features, no single one of which is indispensable or solely responsible for their desirable attributes. Without limiting the scope of the claims, some of the advantageous features will now be summarized.

Herein are disclosed systems and methods for determining the response of bacterial and fungal cultures to antimicrobials by measuring properties of individual microbes and/or other culture particles. Methods include incubating microbes in liquid cultures which may contain antibiotics and growth media, detecting individual microbes and particles, and measuring properties, which in particular embodiments include the mass of the individual microbes and particles. Data, such as the masses of a plurality of individual microbes or particles, may be collected after an incubation duration. These data may be analyzed to determine susceptibility of microbes contained in the culture to one or more antibiotics. Measurement may be performed after incubation durations shorter than those required to produce aggregate growth of the culture, and analysis may include assessment of metrics other than growth of the culture. The method enables measurement of susceptibility at times prior to times needed for sufficient microbe replication as required by many existing techniques. It also enables accurate results with fewer cultures and minimal culture sample volumes. Systems configured to employ these teachings are also disclosed herein.

In a first aspect, a method may be provided to determine the susceptibility of microbes to antimicrobial agents; producing one or more cultures by suspending microbes in a growth medium, with at least one culture containing an antibiotic at specified concentration, incubating the cultures for a time duration and at a temperature that encourages microbe growth, measuring the contents of each culture by detecting a plurality of individual microbes and particles and measuring at least one property of the individual microbes and particles to produce a population profile containing information about variations in the properties among the individual microbes, and analyzing the population profile data to produce a susceptibility result.

In one embodiment of the first aspect, microbes and particles may be measured using a suspended microchannel. In another embodiment of the first aspect, the property measured may be the mass of the microbe or particle. In one embodiment of the first aspect, microbes and particles may be measured using at least one of a flow cytometer or high-resolution photography/videography. In another embodiment of the first aspect, the property measured may be at least one of the size, shape, or volume of the microbe or particle.

In one embodiment of the first aspect, the incubation time duration may be less than 10 hours. In another embodiment of the first aspect, the microbes may be at least one of bacteria or fungi and the antimicrobial agent may be at least one of an antibiotic or an antifungal. In one embodiment of the first aspect, the susceptibility result may be a minimum inhibitory concentration of the antibiotic.

In another embodiment of the first aspect, the susceptibility result may be a categorial result in one embodiment of the first aspect, the analysis may be one or more statistical measures of the population profile in another embodiment of the first aspect, the analysis may use machine learning.

In a second aspect, system may be provided to determine the susceptibility of microbes to antimicrobial agents, comprising a measurement element to measure properties of microbial cultures, configured to; produce at least one culture of suspended microbes in a growth medium, with at least one culture containing an antibiotic at specified concentration; incubate the cultures and at a temperature known to be suitable for microbe growth; measure the contents of each culture the contents of each culture by detecting a plurality of individual microbes and particles and measuring at least one property of the individual microbes and particles to produce a population profile containing information about variations in the properties among the individual microbes; and analyze the population profile data to produce a susceptibility result.

In one embodiment of the second aspect, the measurement element may include a suspended microchannel. In another embodiment of the second aspect, the property measured may be the mass of the microbe or particle. In one embodiment of the second aspect, the measurement element may include at least one of a flow cytometer or high-resolution photography/videography. In another embodiment of the second aspect, the property measured may be at least one of the size, shape, or volume of the microbe or particle

In one embodiment of the second aspect, the incubation time duration may be less than 10 hours. In another embodiment of the second aspect, the microbes may be at least one of bacteria or fungi and the antimicrobial agent may be at least one of an antibiotic or an antifungal. In one embodiment of the second aspect, the susceptibility result may be a minimum inhibitory concentration of the antibiotic. In another embodiment of the second aspect, the susceptibility result may be a categorial result.

In one embodiment of the second aspect, the analysis may be of one more statistical measures of the population profile. In another embodiment of the second aspect, the analysis may use machine learning.

### BRIEF DESCRIPTION OF THE DRAWINGS

Aspects and advantages of the embodiments provided herein are described with reference to the following detailed description in conjunction with the accompanying drawings. Throughout the drawings, reference numbers may be re-used to indicate correspondence between referenced elements. The drawings are provided to illustrate example embodiments described herein and are not intended to limit the scope of the disclosure.

FIG. 1 shows a basic antimicrobial susceptibility test.

FIG. 2 shows an antimicrobial susceptibility test that produces a minimum inhibitory concentration (MIC) result.

FIG. 3A shows a suspended microchannel based system used to assess culture growth by measuring microbe counts or total biomass as shown in FIG. 3B.

FIGS. 4A and 4B shows how a susceptibility test that measures growth and uses short incubation times can underestimate the MIC compared to longer incubation times.

FIG. 5A shows a suspended microchannel used to produce a population profile showing the distribution of masses of individual microbes as shown in FIG. 5B.

FIG. 6 is a graph showing actual population profiles of microbe masses for increasing concentrations of an antimicrobial.

FIGS. 7A and 7B are graphs that (top) show cultures that increasingly delay their growth by replication at increasing antimicrobial concentrations and that (bottom) increase their mean masses in a short (˜3 hour) timeframe for all antimicrobial concentrations.

### DETAILED DESCRIPTION OF ILLUSTRATIVE EMBODIMENTS

The embodiments described herein are directed to methods and systems capable of providing rapid and accurate antimicrobial susceptibility testing of patient samples containing infectious bacteria or fungi. These tests determine which antibacterial or antifungal drugs will be effective for patient treatment. The teachings of the disclosure address an improvement in diagnostic testing by providing assessment of antibiotic efficacy in time frames much shorter than currently achievable.

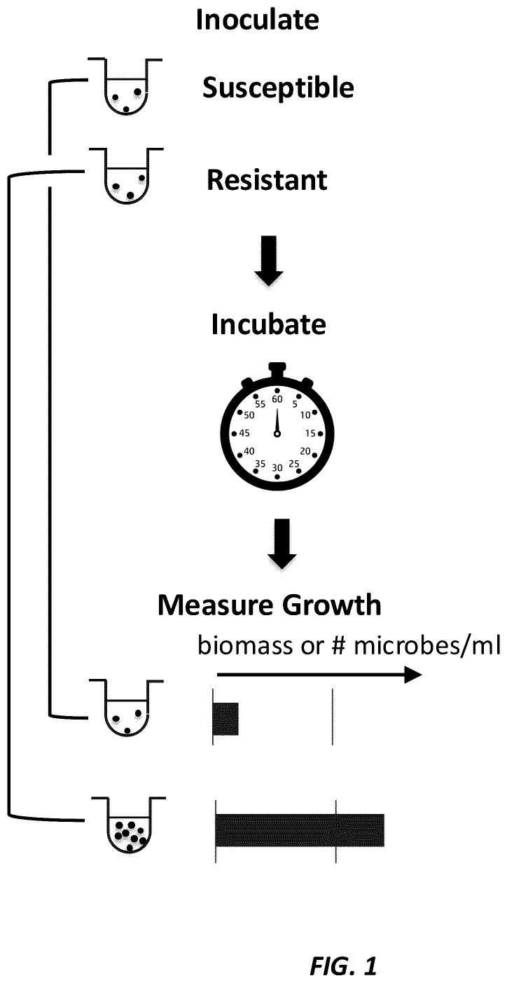

Elements of this disclosure use teachings from U.S. Pat. No. 9,835,614B2, issued Dec. 12, 2017, and owned by the applicant of the current disclosure, incorporated by reference in its entirety, which discloses the basic format and system elements to perform an antibiotic susceptibility test (AST) shown in FIG. 1. Such systems may include an element to present multiple cultures, such as a microtiter well plate, to a measurement element usually under some sort of automated control and analysis elements not described in detail in the current disclosure.

In a typical AST process, infectious bacteria are extracted from a patient sample (for example, infected blood, urine, or other bodily sources) and suspended in a liquid nutrient broth to produce a culture. The broth may contain an antibiotic. The culture is incubated to encourage microbial growth. After a period of time the culture is measured to determine if the culture grew, i.e., in comparison to the culture at the starting point, whether the microbes have replicated, and whether their aggregate biomass (the sum of the microbe masses) has increased. If the culture growth has been suppressed (i.e., there has been little or no replication or increase in biomass), the microbes are classified susceptible to the antibiotic at the tested concentration. If instead the microbes have replicated and their biomass has increased, the microbes are said to be resistant to the antibiotic at the tested concentration. Similar tests can be configured for fungal infections to test the efficacy of antifungal drugs.

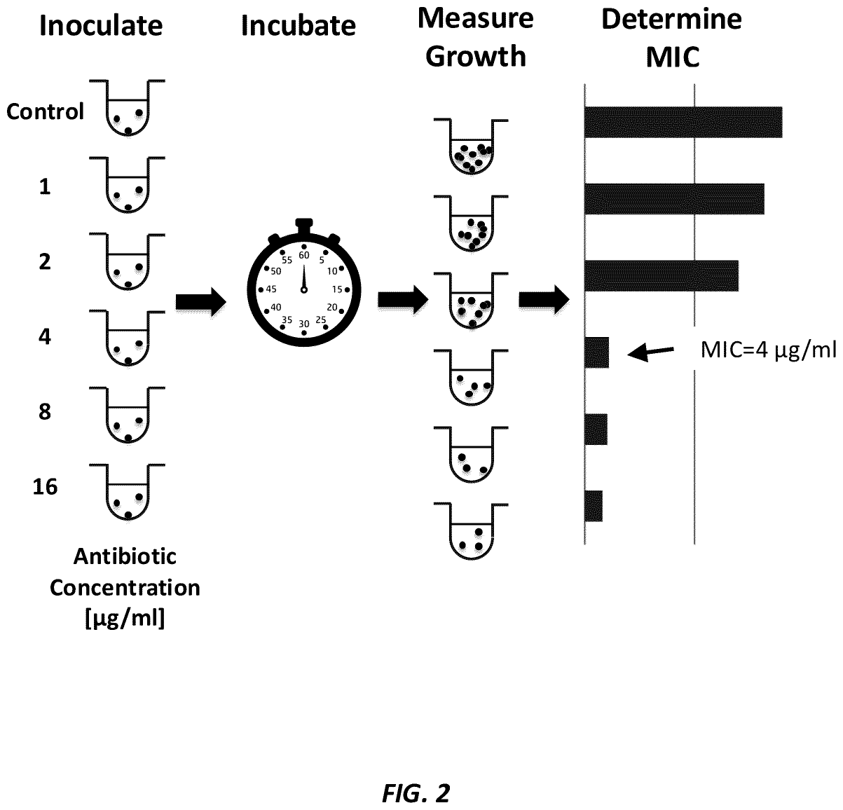

As shown in FIG. 2, implementations of this test usually incubate the microbes in multiple culture wells containing the antibiotic at increasing concentrations, typically increasing 2-fold, for example, in each successive well, and may include a “control” culture containing no antibiotic (FIG. 2). In this disclosure, the terms microbial and antimicrobial may be used to describe various types of microbes and agents that suppress their growth, including bacteria, fungi and others.

During the incubation period the cultures grow when the microbes undergo replication, increasing their numbers significantly. This growth is measured for each well, for example by measuring the increase in total biomass in the culture, i.e., the total mass embodied in the increased number of microbes. The minimum concentration of antibiotic that suppresses growth is determined as shown. This “minimum inhibitory concentration” (MIC) is the key result of the AST and provides actionable information as to whether the antibiotic should be prescribed for treatment.

The most reliable, “gold standard” ASTs use the above format with incubation times in the range of 18-24 hours. This gives the cultures ample time to grow and allows growth to be easily measured by observing the turbidity from the robustly growing cultures, usually visible to the naked eye. The clinical efficacy of antibiotics prescribed on the basis of such ASTs is well established.

In addition to the MIC, AST methods can also report a “categorical” result, i.e., selecting one of Susceptible, Intermediate, or Resistant (S/I/R). These are standard, epidemiologically-determined categories reported by ASTs that classify the in-vitro response of a given species of microbe to a particular antibiotic as that best correlating to the efficacy of the antibiotic in-vivo. It would be desirable to produce AST results in much less time than that required by using the long (18-24 hour) incubation times. This is because in some infection scenarios patients can decline rapidly in the absence of effective treatment and the AST result is often needed to guide treatment. To address these scenarios, “rapid” ASTs have been developed which aim to produce a result in a faster timeframe. These ASTs incubate the cultures for a shorter time, and by using sensitive means for measuring culture growth, they attempt to produce an AST result in just a few hours. Many bacteria replicate with a doubling time of less than an hour and as short as 20 minutes. In principle, a sufficiently sensitive measure of growth would determine susceptibility or resistance, or MIC, in a shorter timeframe than the long incubation times used by “gold standard” methods.

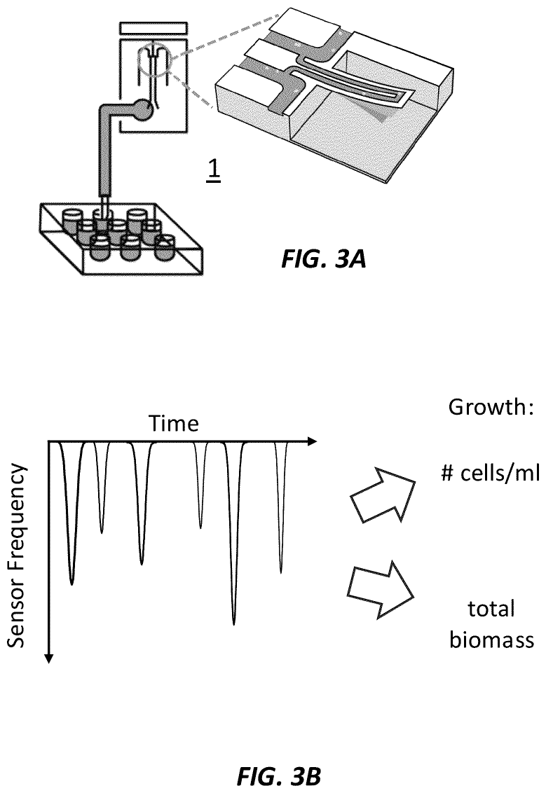

The above incorporated reference discloses such an approach. It uses the basic AST format of FIGS. 1 and 2. To measure the growth in the cultures, an aliquot of each culture is passed through a measurement element which in this particular example includes a mechanically resonating structure such as a suspended microchannel 1 of appropriate size (FIGS. 3A and 3B). Microbes are at a low enough concentration, and the sensor volumes are small enough, that microbes usually pass through the sensor one at a time. When a microbe passes though the sensor, the microchannel's resonant frequency is changed in proportion to the mass of the microbe. This effect is described in the art as a method of measuring particle characteristics such as mass and size. Such methods of measuring particle characteristics such as mass and size are described in U.S. Pat. No. 8,087,284, and related applications, incorporated in their entirety by reference. The referenced teachings form the basis for certain commercial particle measurement instruments.

By counting the microbes so detected, and/or by adding their masses so measured, a measure of the growth in the culture is produced. This method has been implemented in a commercial AST system and can detect and measure growth after shorter incubation times than conventional methods. Other measurement methods based on sensitive detection of culture growth have also been developed. Example measurement approaches and elements include sensitive optical measurements, analysis of high-resolution images or videos of cultures, flow cytometry, measuring redox byproducts produced in culture growth, and detecting volatile gases produced by the cultures.

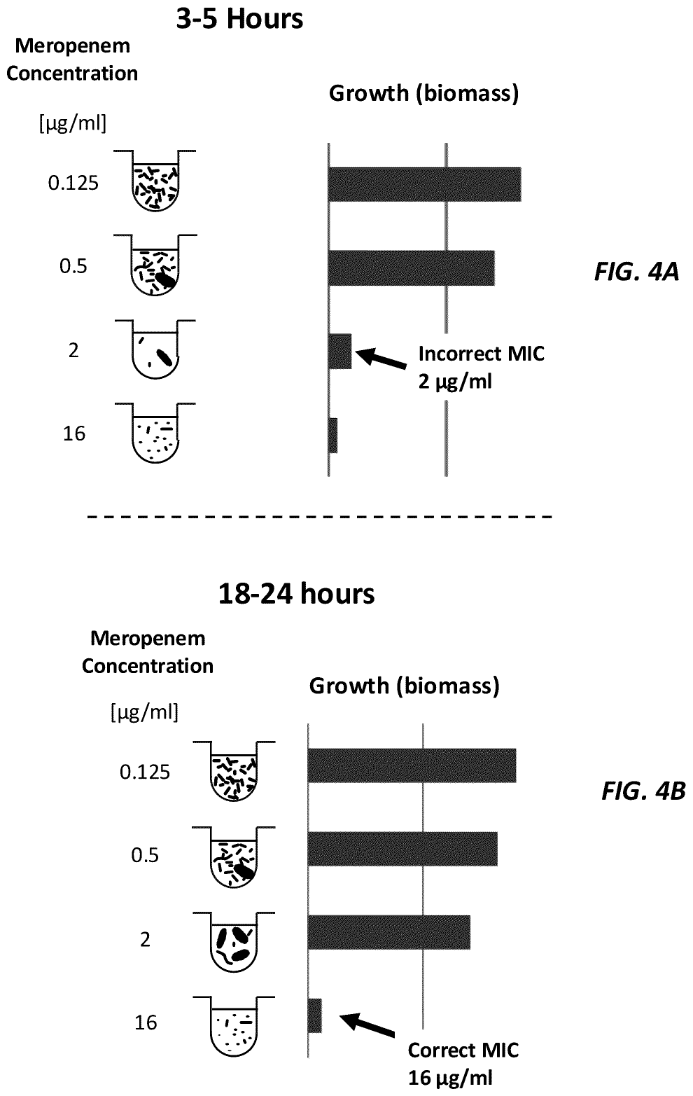

However, it has been found that, despite improved sensitivity in measuring culture growth, these rapid ASTs do not produce accurate results in some cases. FIGS. 4A and 4B show an example of cultures of the same microbes incubated for different times. The cultures in FIG. 4A were incubated for a short time (3-5 hours), and in FIG. 4B for the longer 18-24-hour interval. For the shorter time, the MIC is indicated to be 2 μg/ml of the antibiotics. Whereas, waiting for the longer incubation time results in an MIC of 16 μg/ml. That is, the AST results for the longer incubation time indicate that a much higher concentration of the antibiotic is needed to inhibit growth in the culture. As described earlier, it is the results for these long incubation times that are the diagnostic standard and have shown to best indicate in-vivo efficacy in patient treatment. The anomalously low MIC value of the rapid (short incubation time) may erroneously indicate that the microbe growth is suppressed by the antibiotic when in fact it may be resistant in-vivo and lead to ineffective treatment—a dangerous error.

In FIGS. 4A and 4B, the microbes in the lowest concentrations of antibiotic replicate in the shorter incubation timeframe (see wells with 0.125 and 0.5 μg/ml of antibiotic). However, the microbes in the well containing 2 μg/ml do not replicate in this short time but do replicate after the extended 18-24-hour incubation time. That is, the microbes in this culture *delay* their growth. This behavior is observed for many infectious microbes in response to a number of different antibiotics. The biological mechanisms that cause this behavior are the subject of ongoing research, but the resultant effect complicates the ability to determine MIC in reduced timeframes

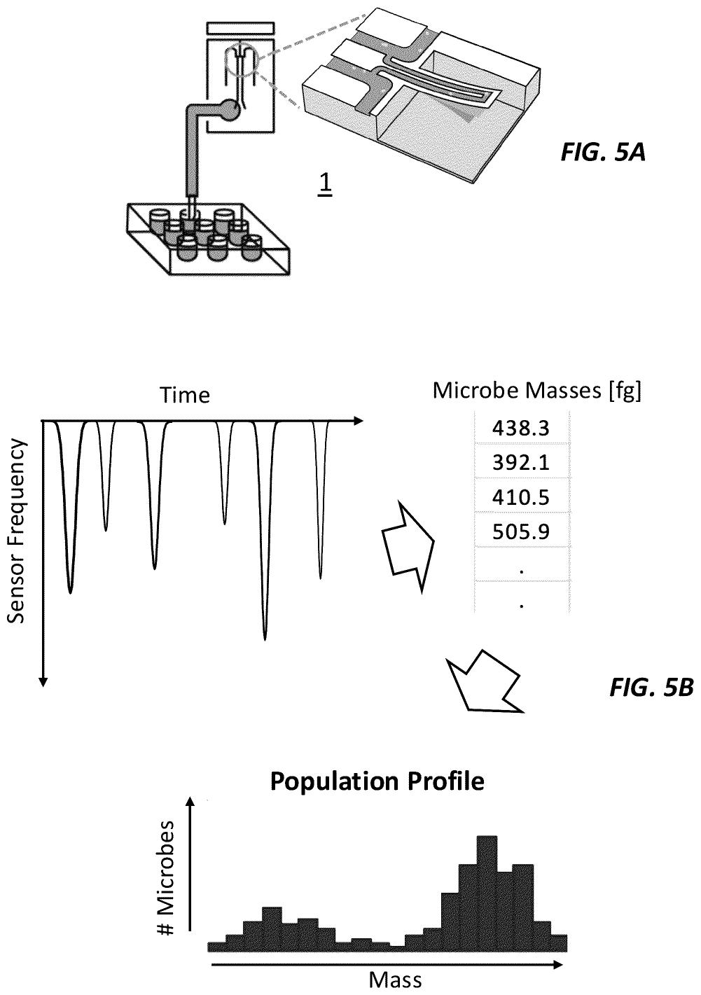

The current disclosure teaches a system and method for producing correct AST results at short incubation times even when confronted with cultures that delay their growth as illustrated in FIG. 4. It can also improve accuracy when measuring cultures that do not delay their growth. FIGS. 5A and 5B shows one embodiment, in which the measurement element, a suspended microchannel, l is used to measure the masses of individual microbes or other particles in the culture such as microbe fragments or other byproducts, FIG. 5B. The raw data can be represented as a histogram to show how the particle masses are distributed among different mass ranges. This data set constitutes a “population profile” of the culture. In comparison to FIG. 3, it contains more information than aggregate measures of culture growth such as the number of measured microbes or the sum of their masses. It can be analyzed to produce a correct MIC even when the culture has not yet grown by replication. In fact, acquiring the information in this fashion produces a wholly unanticipated outcome: these population profiles of distributed properties may correlate with AST results produced by “gold-standard” methods, even if no growth has in fact yet taken place.

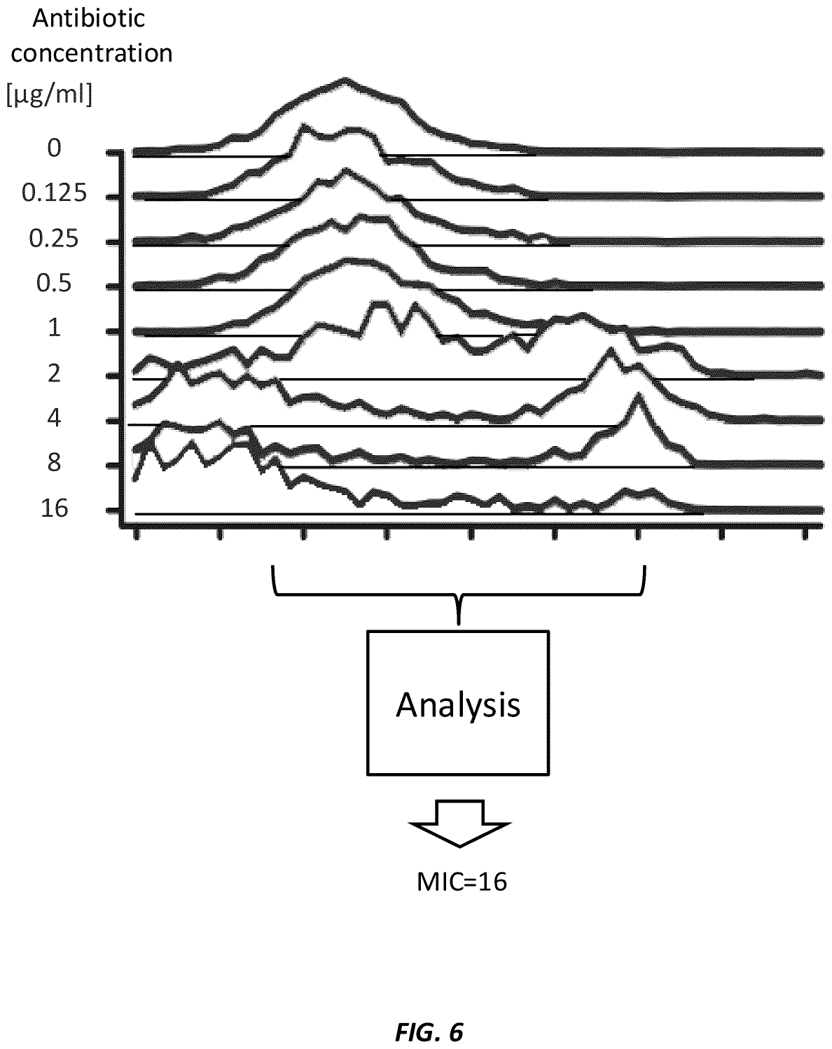

FIG. 6 shows an example of population profiles from an actual sample and susceptibility test, for a range of antibiotics concentrations with the aim of determining the MIC. Compared to measures solely of growth, these measurements contain more information about the response of the microbes to the antibiotic at various concentrations. For example, the population profiles near the top, at lower antibiotic concentrations, show fairly normal mass distributions indicative of healthy, replicating cells and growing cultures. The bottom culture, at the highest antibiotic concentration, shows a large population of low mass debris. The intermediate population profiles, for antibiotic concentrations 2, 4, and 8 μg/ml, show the presence of microbes with abnormally *large* masses compared to the normal cultures. This response may be due to mechanisms in which the antibiotic degrades the cell wall but does not outright kill the cell. The cell's metabolism persists, and it accumulates mass and “blows up” into an abnormally large structure, with an abnormally large mass. Some of these cells may “wait out” the antibiotic and eventually survive and replicate. This is the case for the example in FIG. 6 because the correct MIC, as determined by long-incubation time “gold standard” methods, is known to be 16 μg/ml, while intermediate antibiotic concentration cultures do eventually survive and grow, i.e., the microbes are resistant at these antimicrobial concentrations—an example of delayed growth. Susceptibility measurements based on growth in shorter timeframes, i.e., cell number or total mass (for example, such as in the previous, referenced disclosure) indicate that these cultures were not growing and would underestimate the MIC. In addition to the particular example of FIG. 6, microbes exhibit many other mechanisms of response to antibiotics that can similarly delay their growth. A common example is the interruption of cell division, which also leads to abnormally large masses.

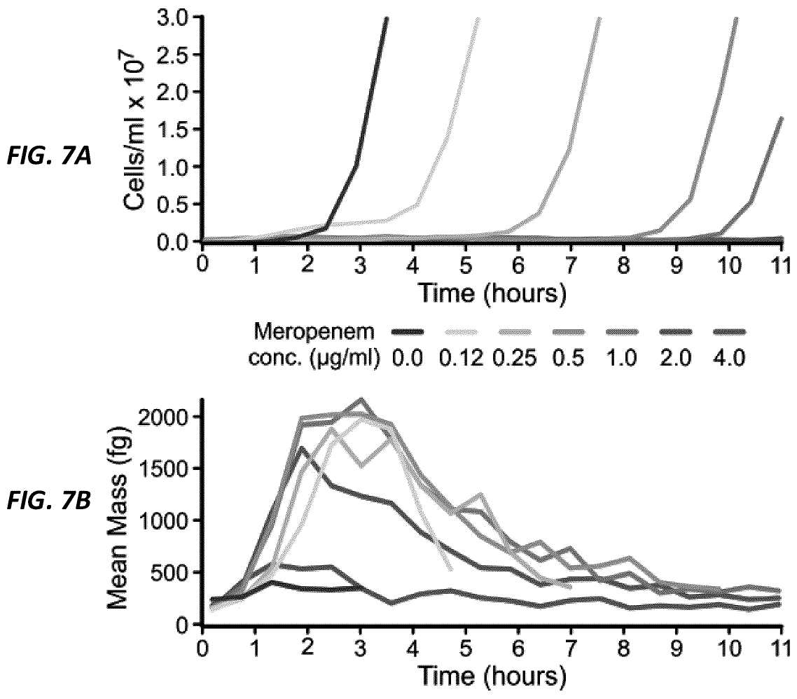

The example in FIG. 6 shows that, for some microbes and antibacterial concentrations, there may be a period where response to the antibacterial agent starts to take place, but replication has not started—a pre-replication period—yet during this period the population profile of the microbe and particle sizes and numbers will have changed from the starting profile. This is illustrated in FIGS. 7A and 7B, which shows measurements of cultures over successive times. FIG. 7A, the top graph shows the number of cells/ml of culture, with an increasing plot indicating replication over time. FIG. 7B, the bottom graph shows the mean of the microbe masses in those cultures. For some antibiotic concentrations, the top graph shows that replication does not occur for the first several hours of incubation. However, the bottom graph shows that for these cultures the mean masses of the microbes have increased prior to the onset of replication, a response to the antibiotic. This information is contained in the population profiles. AST methods that rely on measuring aggregate growth in cultures, such as illustrated in FIGS. 2, 3, and 4, cannot detect these pre-replication changes, even when the detection methods are very sensitive.

These examples show that the population profile can provide a signature of pre-replication properties and response to an antibiotic, and, with subsequent analysis, enable MIC results that agree with those derived from long-term, “gold Standard” growth measurements. As shown in FIG. 7, pre-replication time period is typically short compared to those longer-term methodologies. Although this pre-replication time may vary for different microbes and agents, surveys of numerous samples of different bacteria species and strains show this period to typically be in the range of 1-8 hours. Population profile measurements may best be employed in this range of incubation times but need not be limited to it.

A number of approaches can be used to analyze these population profiles to produce a susceptibility result. For example, the presence of sub-normal mass debris may be a sign of susceptibility. The mean mass of the microbes may be calculated from the population profiles and indicate the presence of normal vs. abnormal cell forms such as spheroplasts or filaments caused by a response an antibiotic. A more detailed statistical analysis of the population profiles can determine, for example, the modes (peaks) in the distributions, along with their ranges, and similarly detect such responses. And other statistical measures can be applied to the mass distributions embodied in the population profiles.

Machine learning approaches can also be used which enable correlating long term MIC results with shorter term culture population profiles for a large number of bacteria and antibacterial agents without the need to analyze the exact microbiological processes behind the correlation. For example, deep learning neural networks can be trained using standard methods to produce an accurate AST result. Such algorithms may include, among others, Multi-Layer Perceptrons (MLPs), Convolutional Neural Networks (CNNs), and Transformer models. The choice of architecture is determined based on the nature and complexity of the data involved in the analysis.

Such a network is “trained” by introducing population profile data from many samples to the network along with the corresponding known, correct susceptibility results (in most cases, the MICs produced using the “gold standard” AST methods). Using “machine learning” algorithms the connectivity between the internal in-silicon “neurons” is adjusted such that, when a new data set from an unknown sample is introduced, it can produce a correct MIC. These neural networks can be trained on the population profiles for a range of antibiotic concentrations. They can also include data obtained for antibiotics other than the target antibiotic in order to take advantage of information gleaned from the activity of various antibiotics on a given microbe strain.

The response of a given microbe culture, and the resulting population profiles, will vary depending on the antibiotic tested. Different classes of antibiotics affect microbes differently. For example, some compromise the cell wall while others inhibit ribosomal activity. In addition, response to a given antibiotic can vary depending on the microbe's genus or species, and wide variation can be seen from strain to strain. Among these numerous combinations, microbes have developed an enormous number of resistance mechanisms and responses to the various antibiotics. Analyses can be developed that are specific to a given antibiotic or to a given combination of antibiotic and microbe genus and/or species.

Methods based on detecting individual microbes and measuring their individual masses to produce population profiles, when combined with appropriate analyses have been found to be superior to rapid methods based on bulk growth measurements. This assessment results from comparing the accuracy of these rapid methods by comparing their results to the “gold standard”, long-incubation time susceptibility tests. Comparing results for hundreds of bacteria strains and multiple antibiotics, the rapid population profile method agrees with the “gold standard” results in a significantly higher percentage of cases than rapid method based on bulk growth.

Such measurements can be directly implemented using the resonating microchannels described above. The examples shown focus on mass as the property measured for each microbe. It would also be possible to use measures of other properties such as surface area; or some measure of the metabolism of an individual microbe that could, for example, be revealed by a chemical label; or many others. Population profiles analogous to those using mass could then be analyzed to determine susceptibility. In addition to a suspended microchannel, other methods and systems could be employed to produce population profiles. Examples include flow cytometry, which provides a measure of individual cell volume, and high-resolution imaging or videography capable of resolving individual cells.

In sum, implementations of the population profiling system and method described here have demonstrated superior performance to susceptibility tests based only on growth. Elements contributing to the accuracy of this method are the ability to measure properties of individual microbes and other associated particles and to analyze the resulting population profiles. This allows the correct susceptibility result to be determined rapidly, using short incubation times even in cases when the cultures may not have begun to replicate. The method also requires smaller volumes of culture than are needed than with approaches that measure growth, a benefit when there is limited sample available from the patient. For many cases, measurements need only be performed at a single incubation time point (an “endpoint” measurement), a desirable simplification compared to the plurality of time points described in the above referenced application. In addition, the population profiling method enables the use of fewer antibiotic dilutions to produce a correct susceptibility result than purely growth-based methods, enabling faster measurement times and the ability to produce results for more antimicrobials.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the elements illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

We claim:

1. A method to determine the susceptibility of microbes to antimicrobial agents, comprising:
producing one or more cultures by suspending microbes in a growth medium, with at least one culture containing an antibiotic at specified concentration;
incubating the cultures for a time duration and at a temperature that encourages microbe growth;
measuring the contents of each culture by detecting a plurality of individual microbes and particles and measuring at least one property of the individual microbes and particles to produce a population profile containing information about variations in the properties among the individual microbes;
analyzing the population profile data to produce a susceptibility result.

2. The method of claim 1 of claim 1 wherein microbes and particles are measured using a suspended microchannel.

3. The method of claim 2 wherein the property measured is a mass of the microbe or particle.

4. The method of claim 1 wherein microbes and particles are measured using at least one of a flow cytometer or high-resolution photography/videography.

5. The method of claim 1 wherein the property measured is at least one of size, shape, or volume of the microbe or particle.

6. The method of claim 1 wherein the incubation time duration is less than 10 hours.

7. The method of claim 1 wherein the microbes are at least one of bacteria or fungi and the antimicrobial agent is at least one of an antibiotic or an antifungal.

8. The method of claim 1 wherein the susceptibility result is a Minimum Inhibitory Concentration of the antibiotic.

9. The method of claim 1 wherein the susceptibility result is a categorial result

10. The method of claim 1 wherein the analyzing step comprises one or more statistical measures of the population profile

11. The method of claim 1 wherein the analyzing step comprises machine learning.

12. A system to determine the susceptibility of microbes to antimicrobial agents, comprising a measurement element to measure properties of microbial cultures, configured to;
produce at least one culture of suspended microbes in a growth medium, with at least one culture containing an antibiotic at specified concentration;
incubate the cultures and at a temperature known to be suitable for microbe growth;
measure the contents of each culture the contents of each culture by detecting a plurality of individual microbes and particles and measuring at least one property of the individual microbes and particles to produce a population profile containing information about variations in the properties among the individual microbes;
analyze the population profile data to produce a susceptibility result.

13. The system of claim 13 wherein the measurement element includes a suspended microchannel.

14. The system of claim 14 wherein the property measured is a mass of the microbe or particle.

15. The system of claim 13 wherein the measurement element includes at least one of a flow cytometer or high-resolution photography/videography.

16. The system of claim 13 wherein the property measured is at least one of size, shape, or volume of the microbe or particle.

17. The system 13 wherein the incubation time duration is less than 10 hours.

18. The system of claim 13 wherein the microbes are at least one of bacteria or fungi and the antimicrobial agent is at least one of an antibiotic or an antifungal.

19. The system of claim 13 wherein the susceptibility result is a Minimum Inhibitory Concentration of the antibiotic.

20. The system of claim 13 wherein the susceptibility result is a categorial result

21. The system of claim 13 wherein the analysis comprises one or more statistical measures of the population profile.

22. The system of claim 13 wherein the analysis comprises machine learning.
